(19) United States
(12) Patent Application Publication
(10) Pub. No.: US 2007/0005071 A1
(43) Pub. Date: Jan. 4, 2007

(54) System and method for locating resorbable tissue fixation devices

(75) Inventor: Theodore R. Kucklick, Los Gatos, CA (US)

Correspondence Address: Crockett & Crockett, 24012 Calle De La Plata, Suite 400, Laguna Hills, CA, 92653, US

(73) Assignee: Cannuflow, Inc.

(21) Appl. No.: 11/173,328

(22) Filed: Jun. 30, 2005

(51) Int. Cl. A61B 17/58

(52) U.S. Cl. 606/76

(57) ABSTRACT

A system and method is disclosed for locating resorbable plastic tissue fixation devices during arthroscopic surgery through the use of fluorescence or phosphorescence.

### FIELD OF THE INVENTIONS

The inventions described below relate to the field of surgical instruments.

### BACKGROUND OF THE INVENTIONS

Arthroscopy is a surgical procedure surgeons use to visualize, diagnose and treat problems within a joint of a patient. Arthroscopic surgery involves using optical instruments, such as an arthroscope, to visualize an operating field inside or near a joint of a patient. The same instrument or other instruments may be used to perform a surgical procedure in the operating field. Common instruments used in addition to the arthroscope include a trimming instrument for cutting tissue and an irrigation instrument for irrigating the surgical field.

During many arthroscopic surgical procedures, resorbable tissue fixation devices (staples, clips, screws, etc.) are required. Resorbable tissue fixation devices are used to attach cartilage and ligaments to bone as well as for tissue repair during surgery. They are made from a material that resorbs biologically over time in a patient. Use of resorbable tissue fixation devices reduces the need for follow-up surgery to remove metal screws or staples and the risk of additional complications to the patient.

Though many advances have been made in the development of resorbable tissue fixation devices, several issues remain with their use in arthroscopic surgery. While using these devices during a surgical procedure, the surgeon must verify the manner of placement and number of resorbable tissue fixation devices being implanted. Surgeons must avoid unnecessary protrusion of these devices into a joint and must place these devices properly to effectively secure the tissue. Surgeons must also use the appropriate number of resorbable tissue fixation devices to facilitate safe and effective healing.

The verification process can be difficult and time consuming. Due to the nature of surgery, the view of the surgical field through an arthroscope can become blurred and foggy due to blood and debris. This can make it difficult to locate resorbable tissue fixation devices in the surgical site and evaluate their manner of placement and numbers. Further, pieces of resorbable tissue fixation devices may break off during surgery and need to be retrieved from the surgical site. A surgeon must view the surgical site several times with the arthroscope during a surgical procedure to evaluate the location and placement of fixation devices. Due to the difficulty in viewing resorbable tissue fixation devices during arthroscopoic surgery, a safe and effective system and method are required to properly evaluate the fixation devices' manner of placement.

### SUMMARY

The systems and methods illustrated below provide for locating resorbable plastic tissue fixation devices through the use of fluorescence or ultraviolet light. In the system describe below, resorbable plastic tissue anchors are made from materials having fluorescent or phosphorescent properties. A wand that emits short wavelength light is used to highlight the resorbable tissue fixation devices used at the surgical site. This highlighting makes it easier for the surgeon to locate the fixation devices during surgery and evaluate their manner of placement.

### BRIEF DESCRIPTION OF THE DRAWINGS

FIG. 1 shows a method of performing arthroscopic surgery on a patient.

FIG. 2 shows a detailed view of a typical arthroscopic procedure requiring resorbable tissue fixation devices used in the fixation device locating system.

FIG. 3 illustrates a resorbable tissue fixation device in the shape of a staple.

FIG. 4 shows an external illumination device of the fixation device locating system having an internal power source.

FIG. 5 shows an external illumination device of the fixation device locating system with an electrical cord and an external power source.

FIG. 6 illustrates the tissue fixation device locating system in use.

### DETAILED DESCRIPTION OF THE INVENTIONS

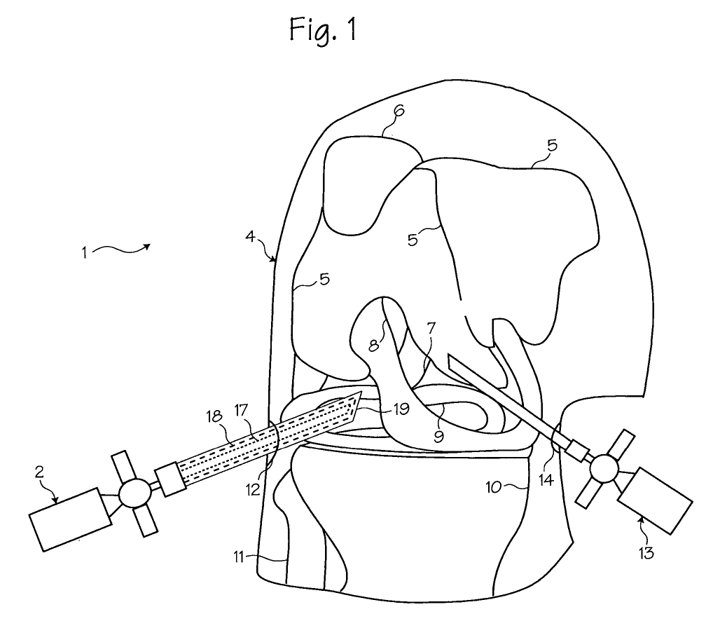

FIG. 1 shows a method of performing arthroscopic surgery on a patient 1 by using an arthroscopic instrument 2 sheathed in an atraumatic introducer sheath 3. An arthroscopic instrument may be an athroscope, endoscope, awl, pick, shaver, etc. In FIG. 1, the arthroscopic instrument 2 shown is an arthroscope. (The various parts of the arthroscope are shown in phantom to indicate their positions inside the sheath.) Various anatomical landmarks in the patient's knee 4 are shown for reference, including the femur 5, patella 6, posterior cruciate ligament 7, anterior cruciate ligament 8, meniscus 9, tibia 10 and fibula 11. During surgery, the surgeon introduces the arthroscope 2 into the knee via a first incision 12 in order to visualize the surgical field. A trimming instrument 13 is introduced through a second incision 14 to remove or trim tissue that the surgeon determines should be removed or trimmed. Optionally, an irrigating instrument may be introduced through a third incision in order to irrigate the surgical field and thereby maintain a clear view.

The arthroscope 2 is an optical instrument 17 surrounded by a rigid cannula 18 having a distal edge that typically is cut at an angle. To protect the patient from unintended injury or trauma during the procedure, the arthroscope has been inserted into a resilient, outer introducer sheath or atraumatic sheath 3 that extends over the rigid cannula. The distal tip 19 of the atraumatic sheath extends distally just past the distal end of the arthroscope and rigid cannula to further protect the patient.

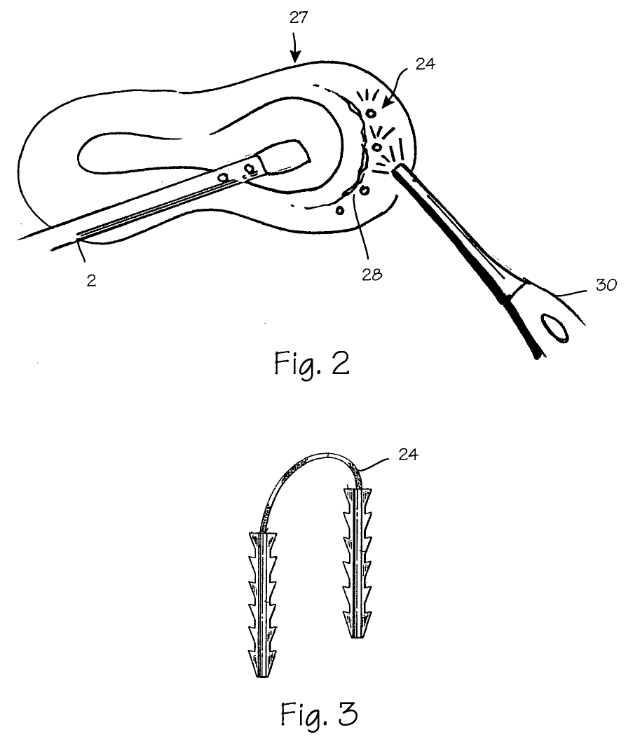

FIG. 2 shows a detailed view of a typical arthroscopic procedure requiring resorbable tissue fixation devices 24 used in the fixation device locating system. Tissue fixation devices can be in the shape of screws, staples, pins, plates, meshes, anchors, arrows, or nails. FIG. 3 illustrates a resorbable polymer tissue fixation device in the shape of staple. Resorbable polymer tissue fixation devices are typically made from materials such as polyglycolic acid (PGA) and polylactic acid (PLA). The body of the patient biologically absorbs these materials and others like them over time.

In FIG. 2, we see an arthroscope 2 and an example of a meniscus 27 tear 28 in the knee of a patient requiring the use of resorbable staples 29. The staples are used to secure the tear in the meniscus. In the tissue fixation device locating system, the fixation devices comprise materials that absorb radiation of first wavelength, or first group of wavelengths, and re-emits photons of a second wavelength or other materials that are capable of emitting light not caused by heat. Materials that are suitable for resorbable tissue fixation devices 24 in the fixation device locating system include luminescent, fluorescent, photochromic, phosphorescent, IR fluorescing, or optically variable pigment materials. The luminescent, fluorescent, photochromic, IR fluorescing or OVP (optically variable pigmentation) properties of the fixation devices may-be enhanced by the use of additives or coatings to the base material. These additives or coatings may include reflective biocompatible metals and oxides, such as gold, titanium dioxide, and silicon dioxide, daylight fluorescing agents, and bioluminescent agents. Bioluminescent agents may include enzymes, biotin, avidin, steptavidin, digoxygenin, luminescent agents, fluorophores, dyes, haptens, luciferins, and the like. The luminescent agents, depending on the source of excitation energy, may be chemoluminescent, bioluminescent, photoluminescent and phosphorescent agents.

In FIG. 2, the staples are manufactured from a material selected due to its fluorescent properties when exposed to light having a short wavelength such as Ultra-violet light. However, in the tissue fixation device locating system, tissue fixation devices, may also be made from natural or synthetic suture or anchor materials including biocompatible impant material such as polyesters, cotton, linen, silk, polyamides (Nylons), polyesters, fluoropolymers, polyolefins, polyetheretherketones (PEEK), and acrylics (PMMA). Degradable suture or anchor materials may include homopolymers, such as polyglycolides, polylactides, polycaprolactones, polytrimethylene carbonates and polydioxanones, and copolymers, such as polylactide-co-glycolides, polycaprolactone-co-glycolides, polyglycolide-co-trimethylene carbonates, polyalkylene diglycolates, and polyoxaesters and polyhydroxyalkanoates. Bone screws and bone cements having luminescent, fluorescent, photochromic, IR fluorescing properties may also be used in the locating system. Bone screws and bone cements may be manufactured from materials such as calcium phosphates, calcium sulphates, demineralized bone matrix or hydroxyapatite. Once the tissue fixation devices with fluorescent properties have been secured in a surgical site, an illumination device 30 that emits light having a second wavelength is placed in the surgical site to illuminate the tissue fixation devices at a first wavelength. Here, short wavelength light, such as ultraviolet light having a wavelength in the range of 4000-10 Angstroms, is used.

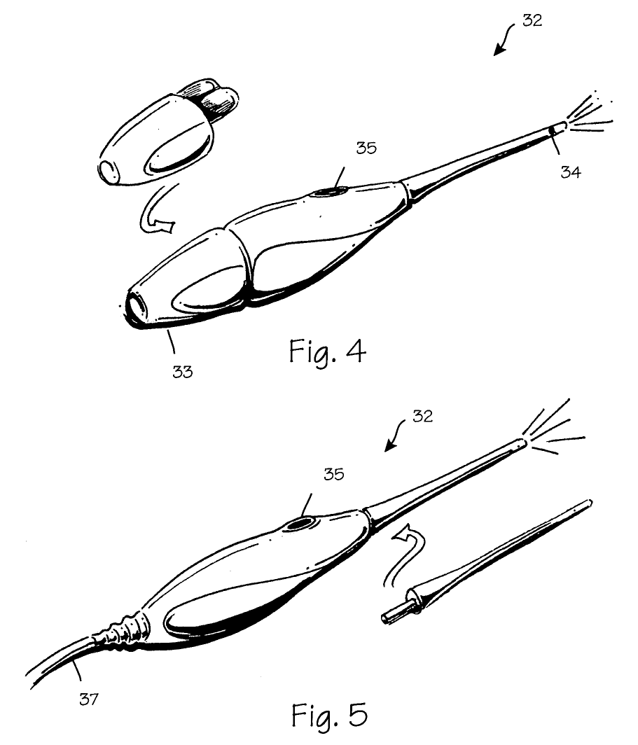

As illustrated in FIG. 4, the illumination device can be a wand 32 comprising a power source 33, LED 34 internal light source and an ON and OFF switch 35. When the wand is switched ON, transmits power to the LED and the LED emits ultraviolet light into the surgical site. The wand may have an internal power source including a battery. The battery can be recharged at recharging station when the wand is coupled to the recharging station. A removable battery can also be provided. When a wand having a removable battery is used, the removable batter may separately be coupled to the recharging station without the wand. In a wand configuration using an internal power source, the wand is cordless allowing easy manipulation by the surgeon or technician during surgery.

Alternatively, as illustrated in FIG. 5, the wand 32 may be provided with an external light source such as a fluorescent lamp or an ultraviolet vapor lamp. The light is communicated from the external light source through an optical cable 37 to the wand. A shutter ON and OFF switch 35 is provided to open the shutter in the wand an allow light to pass into a surgical site or close the shutter to prevent light from passing into the surgical site.

In other configurations of the tissue fixation device locating system, the illumination device may be coupled to an arthroscopic surgical instrument such as an arthroscope or trimmer. In this locating system configuration, light having a short wavelength is emitted within the surgical site via an arthroscopic surgical instrument having a ultraviolet light source incorporated in the instrument. Once the light is emitted, the resorbable polymer tissue fixation devices having fluorescent properties are illuminated by the light source within the surgical device.

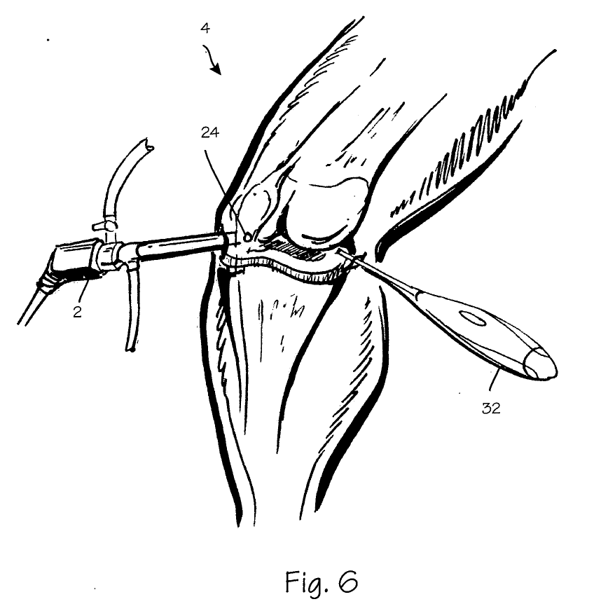

FIG. 6 illustrates the tissue fixation device locating system in use. Here, the patient's knee 4 is the subject of the arthroscopic surgical procedure. An arthrsocope 2 is used by a surgeon to view the surgical site during surgery. As shown, a resorbable polymer tissue fixation 24 device having fluorescent properties is used by the surgeon during the surgical procedure to repair damaged tissue. During the procedure, an illumination wand 32 is inserted within the surgical site. The wand emits light having short wavelengths that can illuminate the resorbable fixation device having fluorescent properties. Once the wand highlights the fixation devices, the devices can easily be located and evaluated for proper placement and orientation by the surgeon or technician.

Thus, while the preferred embodiments of the systems and methods have been described in reference to the environment in which they were developed, they are merely illustrative of the principles of the inventions. Other embodiments and configurations may be devised without departing from the spirit of the inventions and the scope of the appended claims.

We claim:

1. A tissue fixation device locating system comprising:
a tissue fixation device comprising a resorbable polymer having fluorescent properties, said tissue fixation device able to illuminate at a first wavelength when exposed to light having a second wavelength; and
an illumination source able to emit light having the second wavelength.

2. The system of claim 1 wherein the tissue fixation device is in a shape selected from a group of shapes comprising screws, staples, pins, plates, meshes, anchors, arrows, and nails.

3. The system of claim 1 wherein the illumination source is a wand comprising an LED internal light source.

4. The system of claim 3 wherein the wand further comprises an internal power source comprising a battery.

5. The system of claim 3 wherein the wand further comprises an external power source.

6. The system of claim 1 wherein the illumination source is a wand comprising an external light source, a shutter, and an optical cable.

7. A method for locating tissue fixation devices during arthroscopic surgery comprising:
providing resorbable polymer tissue fixation device able to emit a first wavelength of light when exposed to a second wavelength of light to a surgical site;
illuminating the resorbable polymer tissue fixation devices using an illumination device emitting the second wavelength of light.

8. The method of claim 7 wherein the surgical site is a site of an arthroscopic procedure.

9. The method of claim 7 wherein the resorbable polymer tissue fixation device is in a shape selected from a group of shapes comprising screws, staples, pins, plates, meshes, anchors, arrows, and nails.

10. The method of claim 7 wherein the resorbable polymer tissue fixation device is manufactured from a material that absorbs into a body of a patient over time.

11. The method of claim 7 wherein the illumination device comprises an internal light source LED.

12. The method of claim 7 wherein the illumination device comprises an external light source and an optical cable.
